(19) United States
(12) Patent Application Publication
(10) Pub. No.: US 2026/0315398 A1
(43) Pub. Date: Oct. 15, 2026

(54) POLYPEPTIDE DRUG-CONTAINING SUSTAINED-RELEASE MICROSPHERE, PHARMACEUTICAL FORMULATION THEREOF, PREPARATION METHOD THEREFOR, AND USE THEREOF

(71) Applicant: HUILLY PHARMA CO., LTD, Suzhou (CN)

(72) Inventors: Yuning Hu, Suzhou (CN); Xin Yin, Suzhou (CN); Ping Xu, Suzhou (CN); Chun Zhang, Suzhou (CN)

(73) Assignee: HUILLY PHARMA CO., LTD, Suzhou (CN)

(21) Appl. No.: 19/685,341

(22) Filed: May 22, 2026

Related U.S. Application Data
(63) Continuation of application No. PCT/CN2024/128376, filed on Oct. 30, 2024.

(30) Foreign Application Priority Data
Nov. 24, 2023 (CN) 2023115857505

(51) Int. Cl. A61K 9/50; A61K 9/00; A61K 9/19; A61K 38/26; A61K 47/02; A61K 47/14; A61K 47/22; A61K 47/26

(52) U.S. Cl. CPC A61K 9/5031; A61K 9/0019; A61K 9/19; A61K 9/5089; A61K 38/26; A61K 47/02; A61K 47/14; A61K 47/22; A61K 47/26

(57) ABSTRACT

Provided are a polypeptide drug-containing sustained-release microsphere, a pharmaceutical formulation thereof, a preparation method therefor, and use thereof. The method includes the following steps: forming an inner aqueous phase; forming an oil phase; forming an outer aqueous phase; dispersing the inner aqueous phase into the oil phase to form a primary emulsion, then redispersing the primary emulsion into the outer aqueous phase to form a pre-double emulsion, and emulsifying the pre-double emulsion to form a double emulsion; and finally solidifying the double emulsion to obtain the sustained-release microsphere. The preparation method is simple to operate, and the obtained sustained-release microsphere exhibits an ideal drug release behavior (with low initial burst release and stable drug release in middle and later stages), which can reduce blood glucose safely and effectively, enable long-term and stable medication administration, and has high patient compliance.

### CROSS-REFERENCE TO RELATED APPLICATION

This application is a Continuation of PCT Patent Application No. PCT/CN2024/128376 filed on Oct. 30, 2024, which claims priority to Chinese Patent Application No. 202311585750.5, filed with the China National Intellectual Property Administration on Nov. 24, 2023 and entitled “Polypeptide drug-containing sustained-release microsphere, pharmaceutical formulation thereof, preparation method therefor, and use thereof”, which is incorporated herein by reference in its entirety.

### TECHNICAL FIELD

The present disclosure belongs to the field of pharmaceutical formulations, and in particular to a polypeptide drug-containing sustained-release microsphere, a pharmaceutical formulation thereof, a preparation method therefor, and use thereof.

### BACKGROUND

Type 2 diabetes mellitus is a disease characterized by hyperglycemia, which is associated with insulin resistance and relative insulin deficiency. Although the type 2 diabetes mellitus is generally managed initially by increased exercise and dietary adjustment, pharmacological treatment is usually required as the disease progresses.

GLP-1 (glucagon-like peptide-1) is incretin hormones naturally present in the human body. The incretin hormones are a group of metabolic hormones for lowering blood glucose levels, which are secreted after meals to enhance insulin secretion from pancreatic β-cells in a blood glucose-dependent manner. GLP-1 exerts corresponding hypoglycemic effects by activating widely distributed GLP-1 receptors (GLP-1R), but it is highly susceptible to degradation in vivo. Liraglutide and semaglutide, developed by Novo Nordisk, are derived from native GLP-1 via amino acid substitution and side-chain modification, which have high homology in amino acid sequence with native human GLP-1, have half-lives of approximately 12 hours and 160 hours, respectively, and are administered by once-daily or once-weekly injection. Glycemic control requires long-term maintenance, whereas long-term drug injection leads to poor patient compliance and low medication accuracy.

Microsphere formulations encapsulating drugs can extend the duration of drug efficacy by virtue of the carrier material, improve patient compliance, optimize therapeutic effects, and enhance medication accuracy. The microsphere formulations have been widely applied in the development of long-acting sustained-release injections. At present, application scope of five categories of microsphere formulations marketed in China include malignant tumors, diabetes mellitus, cardiovascular diseases, mental illnesses, and other conditions. With expanding population of patients with chronic diseases, the demand for related drugs is vast. Therefore, developing and applying microsphere formulations with significant advantages in the field of chronic diseases can effectively improve health and medication experience of the corresponding patient groups and greatly enhance the accuracy of relevant clinical medication.

To overcome short in vivo half-life of the native GLP-1, various strategies for half-life extension of drugs have been employed in the synthesis of GLP-1 receptor agonist molecules, where a strategy involving fatty acid acylation at specific amino residues is employed in liraglutide and semaglutide. The fatty acid side chain is used to promote molecular self-association, thereby prolonging diffusion time of the drug from an injection site. In addition, the fatty acid can reversibly and non-covalently bind to albumin to slow renal clearance. As microsphere formulations require encapsulation of a one-month or even multi-month dose of the drug, a high-concentration drug solution needs to be used as an inner aqueous phase during preparation. However, Liraglutide and semaglutide are prone to aggregation and even form drug polymer fibers under high-concentration conditions, where these drug fibers not only cause loss of drug activity but may also lead to surrounding tissue necrosis, posing risks of drug toxicity and immunogenicity^{[1-2]}. Therefore, for the properties of drug substances, how to avoid massive drug aggregation and fibril formation in the long-acting sustained-release formulations remains a major challenge.

In addition, due to amphipathic nature of liraglutide and semaglutide molecules, a large amount of drug is dispersed at an oil-water interface during emulsion preparation. With rapid volatilization of the solvent in the curing process, extensive coalescence of primary emulsion droplets is caused, and the drug tends to migrate to an outer shell surface of the microsphere during this process, which leads to excessively fast release of the microspheres during an initial stage, a high Cmax, consequently affecting safety, and also makes it difficult to maintain long-term stable drug release from the sustained-release formulation. How to release active drugs from sustained-release formulations with low burst release and long-term stability is currently a challenge for such amphiphilic drugs in GLP-1R agonists.
[1] Zapadka K L, Becher F J, Gomes Dos Santos A L, Jackson S E, Factors affecting the physical stability (aggregation) of peptide therapeutics[J], Interface Focus, 2017.
[2] Venanzi M, Savioli M, Cimino R, et al., A spectroscopic and molecular dynamics study on the aggregation process of a long-acting lipidated therapeutic peptide: the case of Semaglutide [J], Soft Matter, 2020.

### SUMMARY

### Problem to be Solved by the Present Disclosure

In view of the foregoing problem, the present disclosure is intended to provide a microsphere formulation of a GLP-1R agonist, which has low initial burst release, stable release in the mid-to-later stages, enables long-term administration, offers high patient compliance, and is easy to operate.

### Solution for Solving the Problem

According to a first aspect, the present disclosure provides a preparation method for a polypeptide drug-containing sustained-release microsphere, including the following steps:
mixing an aggregation inhibitor, an emulsion stabilizer A, and water to obtain a mixture, regulating a pH value of the mixture with a pH regulator A, and then adding the polypeptide drug into the mixture to form an inner aqueous phase;
mixing a carrier material, an emulsion stabilizer B, and an organic solvent to form an oil phase;
mixing a surfactant, an osmotic pressure regulator, and water to obtain a mixture, regulating a pH value of the mixture with a pH regulator B to form an outer aqueous phase; and
dispersing the inner aqueous phase into the oil phase to form a primary emulsion, dispersing the primary emulsion into the outer aqueous phase to form a pre-double emulsion, emulsifying the pre-double emulsion to form a double emulsion, and solidifying the double emulsion to obtain the sustained-release microsphere.

As a further improvement of the present disclosure, the polypeptide drug is a peptide GLP-1R agonist, preferably at least one of liraglutide and semaglutide, more preferably semaglutide.

As a further improvement of the present disclosure, the aggregation inhibitor is at least one of sodium caprylate, sucrose, mannitol, sodium 8-(2-hydroxybenzamido)octanoate (SNAC), phenol, benzyl alcohol, ethanol, and trifluoroethanol, preferably sucrose.

As a further improvement of the present disclosure, a weight ratio of the aggregation inhibitor to the polypeptide drug is 1:70 to 50:70, preferably 27:70.

As a further improvement of the present disclosure, the emulsion stabilizer A is at least one of Tween, poloxamer, sodium oleate, and dextran, preferably sodium oleate.

As a further improvement of the present disclosure, a weight ratio of the emulsion stabilizer A to the polypeptide drug is 1:200 to 3:2, preferably 9:1400.

As a further improvement of the present disclosure, the water is at least one of ultra-pure water, double-distilled water, and water for injection, preferably the ultra-pure water.

As a further improvement of the present disclosure, a dosage ratio of the water to the polypeptide drug is 3 mL:10 to 650 mg, preferably 3 mL:70 mg.

As a further improvement of the present disclosure, the pH regulator A is at least one of hydrogen chloride, sodium hydroxide, and phosphate.

As a further improvement of the present disclosure, the pH regulator A is used to adjust a pH value of the mixture to 6.0 to 9.5, preferably 7.6.

As a further improvement of the present disclosure, the organic solvent is at least one of trichloromethane, dichloromethane, ethyl acetate, acetone, and acetonitrile, preferably dichloromethane.

As a further improvement of the present disclosure, a dosage ratio of the organic solvent to the polypeptide drug is 1 to 10 mL:50 mg, preferably 1 mL:7 mg.

As a further improvement of the present disclosure, the carrier material is a biodegradable carrier material.

As a further improvement of the present disclosure, the carrier material is at least one of poly(lactic-co-glycolic acid) (PLGA), poly(lactic acid)-poly(ethylene glycol) copolymer (PELA), polylactic acid (PLA), polyhydroxyalkanoate (PHA), and polycaprolactone (PCL), preferably poly(lactic-co-glycolic acid), more preferably, a ratio of lactic acid to glycolic acid in every 100 parts of monomers is 100 to 50:0 to 50, preferably poly(lactic-co-glycolic acid) in 50:50.

As a further improvement of the present disclosure, a molecular weight of the carrier material is 1000 to 150,000 Da, preferably 20,000 Da.

As a further improvement of the present disclosure, a weight ratio of the carrier material to the polypeptide drug is 3:1 to 100:1, preferably 240:7.

As a further improvement of the present disclosure, the emulsion stabilizer B is at least one of lecithin, soybean phospholipid, oleic acid, and Span, preferably at least one of Span 83 (Sorbitan Sesquioleate) and Span 85 (Sorbitan Trioleate), more preferably Span 85.

As a further improvement of the present disclosure, a weight ratio of the emulsion stabilizer B to the polypeptide drug is 1:20 to 40:20, preferably 3:14.

As a further improvement of the present disclosure, the surfactant is at least one of polyvinyl alcohol (PVA), Tween, sodium dodecyl sulfate (SDS), sodium dodecyl benzene sulfonate (SDBS), and polyoxyethylene octylphenol ether, preferably Tween, more preferably Tween 20.

As a further improvement of the present disclosure, a weight ratio of the surfactant to the polypeptide drug is 1:20 to 40:20, preferably 3:28.

As a further improvement of the present disclosure, the osmotic pressure regulator is at least one of sodium chloride and potassium chloride, preferably sodium chloride.

As a further improvement of the present disclosure, a weight ratio of the osmotic pressure regulator to the polypeptide drug is 1:1 to 100:1, preferably 80:1.

As a further improvement of the present disclosure, the water is at least one of ultra-pure water, double-distilled water, and water for injection, preferably ultra-pure water.

As a further improvement of the present disclosure, a dosage ratio of the water to the polypeptide drug is 0.001 to 10 mL:1 mg, preferably 50 mL:7 mg.

As a further improvement of the present disclosure, the pH regulator B is at least one of hydrogen chloride, sodium hydroxide, and phosphate.

As a further improvement of the present disclosure, the pH regulator B is used to adjust a pH value of the mixture to 6.0 to 9.5 with, preferably 7.8.

As a further improvement of the present disclosure, the dispersion is accomplished by means of at least one of stirring, homogenization, and ultrasonication, preferably homogenization, more preferably high-pressure homogenization.

As a further improvement of the present disclosure, the high-pressure homogenization lasts for 0.5 to 20 min, preferably 2 min.

As a further improvement of the present disclosure, the high-pressure homogenization is carried out at a pressure of 0.2 to 1200 bar, preferably 1 bar.

As a further improvement of the present disclosure, the redispersion is accomplished by means of at least one of stirring and homogenization, preferably stirring.

As a further improvement of the present disclosure, the emulsification is accomplished by means of membrane emulsification.

As a further improvement of the present disclosure, a membrane pore size for the membrane emulsification is 1.0 to 60 μm, preferably 40 μm.

As a further improvement of the present disclosure, the membrane emulsification is carried out at a transmembrane pressure of 1 to 200 kPa, preferably 50 kPa.

As a further improvement of the present disclosure, the solidification is accomplished by means of volatilization or evaporation, preferably reduced-pressure volatilization or thin-film evaporation, and more preferably thin-film evaporation.

As a further improvement of the present disclosure, the thin-film evaporation lasts for 1 to 50 min, preferably 10 min.

As a further improvement of the present disclosure, the thin-film evaporation is carried out at a temperature of 25 to 50 °C, preferably 35 °C

As a further improvement of the present disclosure, a number of cycles for the thin-film evaporation is 1 to 6, preferably 3.

As a further improvement of the present disclosure, the preparation method further includes solidifying the double emulsion, followed by washing and lyophilizing.

According to a second aspect, the present disclosure provides a polypeptide drug-containing sustained-release microsphere, which is prepared by using the foregoing preparation method.

According to a third aspect, the present disclosure provides a pharmaceutical formulation of a polypeptide drug, including the foregoing sustained-release microsphere.

As a further improvement of the present disclosure, the pharmaceutical formulation further includes at least one pharmaceutically acceptable excipient.

As a further improvement of the present disclosure, the pharmaceutical formulation is a sustained-release formulation.

As a further improvement of the present disclosure, the pharmaceutical formulation is an injection.

According to a fourth aspect, the present disclosure provides use of the sustained-release microsphere described above or the pharmaceutical formulation described above in preparation of a drug for preventing and/or treating a disease and/or disorder at least partially associated with GLP-1R.

As a further improvement of the present disclosure, the disease and/or disorder at least partially associated with GLP-1R is diabetes mellitus, weight loss, or non-alcoholic fatty liver disease, preferably type 2 diabetes mellitus, or weight loss.

### Beneficial Effects of the Present Disclosure

The present disclosure has beneficial effects as follows. The preparation method is simple to operate, the prepared sustained-release microsphere and formulation exhibit stable release behavior. Low initial burst release can avoid the risk of hypoglycemia caused by excessively high initial drug concentration of GLP-1R agonists, and stable mid-to later-stage drug release behavior is safe and effective in hypoglycemic effect. The long-acting sustained-release microsphere preparation is suitable for long-term and stable use and can improve patient compliance.

### BRIEF DESCRIPTION OF THE DRAWINGS

FIG. 1 shows optical microscope images of primary emulsions according to Embodiment 1, Comparative Example 1, and Comparative Example 2;

FIG. 2 shows emulsions and solidification processes of the emulsions according to Embodiment 1, Comparative Example 1, and Comparative Example 2;

FIG. 3 is in vitro release profiles of microspheres according to Embodiment 1, Comparative Example 1, and Comparative Example 2;

FIG. 4 is an in vivo PK (Pharmacokinetic) test result of a rat according to Embodiment 1;

FIG. 5 is in vivo PK test results of rats according to Comparative Example 1 and Comparative Example 2;

FIG. 6 is an aggregation status of GLP-1 and an effect of an aggregation inhibitor;

FIG. 7 shows particle size investigation of three repeated batches in Embodiment 1.

### DETAILED DESCRIPTION OF THE EMBODIMENTS

Various exemplary embodiments, features, and aspects of the present disclosure are described below in detail. As used herein, a term “exemplary” means “serving as an example, an embodiment, or an illustration”. Any embodiment described herein as “exemplary” shall not be construed as superior or preferable to other embodiments.

In addition, to better illustrate the present disclosure, numerous specific details are given out in the following specific implementations. Those skilled in the art shall understand that the present disclosure may be implemented without some specific details. In some other instances, methods, means, devices, and steps well known to those skilled in the art are not described in detail, thereby highlighting the gist of the present disclosure.

Unless otherwise specified, all units used in this specification are international standard units. All values and numerical ranges in the present disclosure shall be understood to include systematic errors unavoidable in industrial production.

In this specification, the term “may” includes both possibilities of carrying out a specific treatment and omitting such treatment.

In this specification, references to “some specific/preferred implementations”, “other specific/preferred implementations”, “implementations” and the like mean that specific elements (for example, features, structures, properties, and/or characteristics) described in connection with such implementations are included in at least one implementation described herein, and may or may not be present in other implementations. In addition, it should be understood the elements may be combined in any appropriate manner in a variety of implementations.

In this specification, a numerical range expressed in the form of “value A to value B” shall include the endpoint values A and B.

In this specification, when the terms “normal temperature” and “room temperature” are used, the temperature may range from 10 °C to 40 °C

### Sustained-Release Microsphere for Polypeptide Drug and Preparation Method Therefor

the present disclosure provides a preparation method for a polypeptide drug-containing sustained-release microsphere, including the following steps:

Mix an aggregation inhibitor, an emulsion stabilizer A, and water to obtain a mixture, regulate a pH value of the mixture with a pH regulator A, and then add the polypeptide drug into the mixture to form an inner aqueous phase.

Mix a carrier material, an emulsion stabilizer B, and an organic solvent to form an oil phase.

Mix a surfactant, an osmotic pressure regulator, and water to obtain a mixture, regulate a pH value of the mixture with a pH regulator B to form an outer aqueous phase; and

Disperse the inner aqueous phase into the oil phase to form a primary emulsion, disperse the primary emulsion into the outer aqueous phase to form a pre-double emulsion, emulsify the pre-double emulsion to form a double emulsion, and solidify the double emulsion to obtain the sustained-release microsphere.

The polypeptide drug in the preparation method (in particular, a polypeptide GLP-1R agonist, such as liraglutide or semaglutide) tends to aggregate at a high concentration and even forms polymer fibers, thereby losing biological activity and causing problems including drug toxicity and immunogenicity. Therefore, maintaining a monomeric form of the drug is particularly critical during preparation. In addition, due to amphipathic nature of drug molecules, the drug tends to migrate to an outer shell layer of the microsphere during preparation, which leads to excessively fast drug release at an initial stage, resulting in an excessively high drug concentration in an early stage and failing to achieve long-term sustained release. Accordingly, it is crucial for preparation to prevent extensive dispersion of drug at an oil-water interface.

According to the present disclosure, an aggregation inhibitor is added to the inner aqueous phase to regulate the pH value, thereby controlling drug aggregation, inhibiting drug fibrillization, and avoiding the reduction in drug activity. In addition, emulsion stabilizers are separately added to the inner aqueous phase and the oil phase to regulate a molecular liquid membrane at the oil-water interface of the double emulsion, which may affect distribution of the amphipathic drug in the emulsion, and ensure uniform dispersion of the primary emulsion during the solidification process. This effectively inhibits massive migration of drug molecules caused by volatilization and removal of organic solvents, enables the drug to be uniformly dispersed in the carrier material. In addition, regulating the pH value of the outer aqueous phase can prevent the drug from migrating to an oil-in-water interface of the double emulsion due to isoelectric point factors, thereby effectively reducing burst release of the drug and maintaining stable drug release behavior.

To illustrate the preparation method for a polypeptide drug-containing sustained-release microsphere clearly, the following detailed description is provided by taking a semaglutide sustained-release microsphere as an example. The method is also applicable to active pharmaceutical ingredients of a polypeptide drug (such as liraglutide) that mainly bind to albumin and the GLP-1 receptor in a monomer form after entering the blood.

Further, the present disclosure provides a preparation method for a semaglutide sustained-release microsphere, including the following steps.

Mix an aggregation inhibitor, an emulsion stabilizer A, and water to obtain a mixture, regulate a pH value of the mixture with a pH regulator A, and then add semaglutide into the mixture to form an inner aqueous phase.

Mix a carrier material, an emulsion stabilizer B, and an organic solvent to form an oil phase.

Mix a surfactant, an osmotic pressure regulator, and water to obtain a mixture, regulate a pH value of the mixture with a pH regulator B to form an outer aqueous phase; and

Disperse the inner aqueous phase into the oil phase to form a primary emulsion, disperse the primary emulsion into the outer aqueous phase to form a pre-double emulsion, emulsify the pre-double emulsion to form a double emulsion, and solidify the double emulsion to obtain the semaglutide sustained-release microsphere.

In an implementation of the present disclosure, the aggregation inhibitor in the foregoing preparation method is at least one of sodium caprylate, sucrose, mannitol, sodium 8-(2-hydroxybenzamido)octanoate, phenol, benzyl alcohol, ethanol, and trifluoroethanol, such as, sucrose, mannitol, or a mixture of the two in any proportion.

In an implementation of the present disclosure, the aggregation inhibitor in the foregoing preparation method may be sucrose.

In an implementation of the present disclosure, a weight ratio of the aggregation inhibitor in the foregoing preparation method to the semaglutide may vary within a specific range, for example 1:70 to 50:70.

In an implementation of the present disclosure, the weight ratio of the aggregation inhibitor in the foregoing preparation method to the semaglutide may be 27:70.

In an implementation of the present disclosure, the emulsion stabilizer A in the foregoing preparation method may be at least one of Tween, poloxamer, sodium oleate, and glucan, such as Tween, sodium oleate, or a mixture of the two in any proportion.

In an implementation of the present disclosure, the emulsion stabilizer A in the foregoing preparation method may be sodium oleate.

In an implementation of the present disclosure, a weight ratio of the emulsion stabilizer A in the foregoing preparation method to the semaglutide may vary within a specific range, for example 1:200 to 3:2.

In an implementation of the present disclosure, the weight ratio of the emulsion stabilizer A in the foregoing preparation method to the semaglutide may be 9:1400.

In an implementation of the present disclosure, the water used for the inner aqueous phase in the foregoing preparation method may be at least one of ultra-pure water, double-distilled water, and water for injection, such as ultra-pure water, double-distilled water, or a mixture of the two in any proportion.

In an implementation of the present disclosure, the water used for the inner aqueous phase in the foregoing preparation method may be ultra-pure water.

In an implementation of the present disclosure, a dosage ratio of the water used for the inner aqueous phase in the foregoing preparation method to the semaglutide may vary within a specific range, for example 3 mL:10 to 650 mg.

In an implementation of the present disclosure, the dosage ratio of the water used for the inner aqueous phase in the foregoing preparation method to the semaglutide may be 3 mL:70 mg.

In an implementation of the present disclosure, the pH regulator A in the foregoing preparation method may be at least one of hydrogen chloride, sodium hydroxide, and phosphate, such as hydrogen chloride, phosphate, or a mixture of the two in any proportion.

In an implementation of the present disclosure, the pH regulator A in the foregoing preparation method may be used to adjust a pH value of the mixture within a specific range, for example 6.0 to 9.5.

In an implementation of the present disclosure, the pH regulator A in the foregoing preparation method may be used to adjust the pH value of the mixture to 7.6.

In an implementation of the present disclosure, the organic solvent in the foregoing preparation method may be at least one of trichloromethane, dichloromethane, ethyl acetate, acetone, and acetonitrile, such as dichloromethane, ethyl acetate, or a mixture of the two in any proportion.

In an implementation of the present disclosure, the organic solvent in the foregoing preparation method may be dichloromethane.

In an implementation of the present disclosure, a dosage ratio of the organic solvent in the foregoing preparation method to the semaglutide may vary within a specific range, for example 1 to 10 mL:50 mg.

In an implementation of the present disclosure, the dosage ratio of the organic solvent in the foregoing preparation method to the semaglutide may be 1 mL:7 mg.

In an implementation of the present disclosure, the carrier material in the foregoing preparation method may be a biodegradable carrier material.

In an implementation of the present disclosure, the carrier material in the foregoing preparation method may be at least one of poly(lactic-co-glycolic acid), poly(lactic acid)-poly(ethylene glycol) copolymer, polylactic acid, polyhydroxyalkanoate, and polycaprolactone, preferably poly(lactic-co-glycolic acid), such as poly(lactic-co-glycolic acid), poly(lactic acid)-poly(ethylene glycol) copolymer, or a mixture of the two in any proportion.

In an implementation of the present disclosure, the carrier material in the foregoing preparation method may be poly(lactic-co-glycolic acid).

In an implementation of the present disclosure, a ratio of lactic acid to glycolic acid in every 100 parts of monomers of the carrier material in the foregoing preparation method may vary within a specific range, for example, 100 to 50:0 to 50, such as 100:0, 95:5, 85:15, 75:25, 50:50, or another ratio.

In an implementation of the present disclosure, a ratio of lactic acid to glycolic acid in every 100 parts of monomers of the carrier material in the foregoing preparation method may be 50:50.

In an implementation of the present disclosure, a molecular weight of the carrier material in the foregoing preparation method may vary within a specific range, for example 1,000 to 150,000 Da.

In an implementation of the present disclosure, the molecular weight of the carrier material in the foregoing preparation method may be 20,000 Da.

In an implementation of the present disclosure, a weight ratio of the carrier material in the foregoing preparation method to the semaglutide may vary within a specific range, for example 3:1 to 100:1.

In an implementation of the present disclosure, the weight ratio of the carrier material in the foregoing preparation method to the semaglutide may be 240:7.

In an implementation of the present disclosure, the emulsion stabilizer B in the foregoing preparation method may be at least one of lecithin, soybean phospholipid, oleic acid, and Span, such as soybean phospholipid, Span, or a mixture of the two in any proportion.

In an implementation of the present disclosure, the emulsion stabilizer B in the foregoing preparation method may be Span 83, Span 85, or a mixture of the two in any proportion.

In an implementation of the present disclosure, the emulsion stabilizer B in the foregoing preparation method may be Span 85.

In an implementation of the present disclosure, a weight ratio of the emulsion stabilizer B in the foregoing preparation method to the semaglutide may vary within a specific range, for example 1:20 to 40:20.

In an implementation of the present disclosure, the weight ratio of the emulsion stabilizer B in the foregoing preparation method to the semaglutide may be 3:14.

In an implementation of the present disclosure, the surfactant in the foregoing preparation method may be at least one of polyvinyl alcohol, Tween, sodium dodecyl sulfate, sodium dodecyl benzene sulfonate, and polyoxyethylene octylphenol ether, such as polyvinyl alcohol, Tween, or a mixture of the two in any proportion.

In an implementation of the present disclosure, the surfactant in the foregoing preparation method may be Tween.

In an implementation of the present disclosure, the surfactant in the foregoing preparation method may be Tween 20.

In an implementation of the present disclosure, a weight ratio of the surfactant in the foregoing preparation method to the semaglutide may vary within a specific range, for example 1:20 to 40:20.

In an implementation of the present disclosure, the weight ratio of the surfactant in the foregoing preparation method to the semaglutide may be 3:28.

In an implementation of the present disclosure, the osmotic pressure regulator in the foregoing preparation method may be at least one of sodium chloride, potassium chloride, or a mixture of the two in any proportion.

In an implementation of the present disclosure, the osmotic pressure regulator in the foregoing preparation method may be sodium chloride.

In an implementation of the present disclosure, a weight ratio of the osmotic pressure regulator in the foregoing preparation method to the semaglutide may vary within a specific range, for example 1:1 to 100:1.

In an implementation of the present disclosure, the weight ratio of the osmotic pressure regulator in the foregoing preparation method to the semaglutide may be 80:1.

In an implementation of the present disclosure, the water used for the outer aqueous phase in the foregoing preparation method may be at least one of ultra-pure water, double-distilled water, and water for injection, such as ultra-pure water, double-distilled water, or a mixture of the two in any proportion.

In an implementation of the present disclosure, the water used for the outer aqueous phase in the foregoing preparation method may be ultra-pure water.

In an implementation of the present disclosure, a dosage ratio of the water used for the outer aqueous phase in the foregoing preparation method to the semaglutide may vary within a specific range, for example 0.001 to 10 mL:1 mg.

In an implementation of the present disclosure, the dosage ratio of the water used for the outer aqueous phase in the foregoing preparation method to the semaglutide may be 50 mL:7 mg.

In an implementation of the present disclosure, the pH regulator B in the foregoing preparation method may be at least one of hydrogen chloride, sodium hydroxide, and phosphate, such as hydrogen chloride, phosphate, or a mixture of the two in any proportion.

In an implementation of the present disclosure, the pH regulator B in the foregoing preparation method may be used to adjust a pH value of the mixture may be regulated within a specific range, for example 6.0 to 9.5.

In an implementation of the present disclosure, the pH regulator B in the foregoing preparation method may be used to adjust the pH value of the mixture to 7.8.

In an implementation of the present disclosure, the dispersion in the foregoing preparation method may be carried out by means of at least one of stirring, homogenization, and ultrasonication, for example, the dispersion is carried out by means of stirring, homogenization, or a combination of the two.

In an implementation of the present disclosure, a dispersion method in the foregoing preparation method may be homogenization.

In an implementation of the present disclosure, the dispersion method in the foregoing preparation method may be high-pressure homogenization.

In an implementation of the present disclosure, a duration of the high-pressure homogenization in the foregoing preparation method may vary within a specific range, for example, 0.5 to 20 min.

In an implementation of the present disclosure, the duration of the high-pressure homogenization in the foregoing preparation method may be 2 min.

In an implementation of the present disclosure, a pressure of the high-pressure homogenization in the foregoing preparation method may vary within a specific range, for example, 0.2 to 1200 bar.

In an implementation of the present disclosure, the pressure of the high-pressure homogenization in the foregoing preparation method may be 1 bar.

In an implementation of the present disclosure, the redispersion in the foregoing preparation method may be accomplished by means of at least one of stirring and homogenization.

In an implementation of the present disclosure, the redispersion in the foregoing preparation method may be accomplished by means of stirring.

In an implementation of the present disclosure, the emulsification in the foregoing preparation method may be accomplished by means of membrane emulsification.

In an implementation of the present disclosure, a membrane pore size for the membrane emulsification in the foregoing preparation method may vary within a specific range, for example, 1.0 to 60 μm.

In an implementation of the present disclosure, the membrane pore size for the membrane emulsification in the foregoing preparation method may be 40 μm.

In an implementation of the present disclosure, a transmembrane pressure of the membrane emulsification in the foregoing preparation method may vary within a specific range, for example, 1 to 200 kPa.

In an implementation of the present disclosure, the transmembrane pressure of the membrane emulsification in the foregoing preparation method may be 50 kPa.

In an implementation of the present disclosure, the solidification in the foregoing preparation method may be accomplished by means of volatilization or evaporation.

In an implementation of the present disclosure, the solidification in the foregoing preparation method may be accomplished by means of pressure-reduced volatilization or thin-film evaporation.

In an implementation of the present disclosure, the solidification in the foregoing preparation method may be accomplished by means of thin-film evaporation.

In an implementation of the present disclosure, a duration of the thin-film evaporation in the foregoing preparation method may vary within a specific range, for example, 1 to 50 min.

In an implementation of the present disclosure, the duration of the thin-film evaporation in the foregoing preparation method may be 10 min.

In an implementation of the present disclosure, a temperature of the thin-film evaporation in the foregoing preparation method may vary within a specific range, for example, 25 to 50 °C.

In an implementation of the present disclosure, the temperature of the thin-film evaporation in the foregoing preparation method may be 35 °C.

In an implementation of the present disclosure, a number of cycles of the thin-film evaporation in the foregoing preparation method may vary within a specific range, for example, 1 to 6 times.

In an implementation of the present disclosure, the number of cycles of the thin-film evaporation in the foregoing preparation method may be 3 times.

In an implementation of the present disclosure, the preparation method further includes the following steps: wash and lyophilize the double emulsion after solidifying.

Correspondingly, the present disclosure provides a polypeptide drug-containing sustained-release microsphere, which is prepared by using the foregoing preparation method.

In an implementation of the present disclosure, the polypeptide drug in the sustained-release microsphere may be a polypeptide GLP-1R agonist.

In an implementation of the present disclosure, the polypeptide drug in the sustained-release microsphere may be semaglutide.

Pharmaceutical Formulation of Polypeptide Drug

The present disclosure provides a pharmaceutical formulation of a polypeptide drug, including the foregoing sustained-release microsphere.

In an implementation of the present disclosure, the pharmaceutical formulation may further include at least one pharmaceutically acceptable excipient, such as a solvent, a solubilizer, and a cosolvent.

In an implementation of the present disclosure, the pharmaceutical formulation may be a formulation with a specific drug release behavior, such as a sustained-release formulation.

In an implementation of the present disclosure, the pharmaceutical formulation may be a parenteral dosage form, such as an injection.

### Medical Use of Sustained-Release Microsphere and Pharmaceutical Formulation Thereof

The present disclosure provides use of the sustained-release microsphere described above or the pharmaceutical formulation described above in preparation of a drug for preventing and/or treating a disease and/or disorder at least partially associated with GLP-1R.

In an implementation of the present disclosure, the disease and/or disorder at least partially associated with GLP-1R may be diabetes mellitus, weight loss, or non-alcoholic fatty liver disease.

In an implementation of the present disclosure, the disease and/or disorder at least partially associated with GLP-1R may be type 2 diabetes mellitus or weight loss.

Implementations of the present disclosure are described in detail below with reference to specific embodiments, but those skilled in the art should understand that the following embodiments are only used to illustrate the present disclosure and should not be regarded as limiting the scope of the present disclosure. For specific conditions not specified in the embodiments, the conventional conditions or the conditions suggested by the manufacturer shall be followed. All reagents and instruments used without specifying the manufacturer are standard laboratory products that are commercially available.

### Embodiment 1

90 mg of sucrose and 1.5 mg of sodium oleate are weighed and then dissolved in 10 mL of ultrapure water to obtain a solution, a pH value of the solution is adjusted to 7.6, and 70 mg of semaglutide is weighed and then dissolved in 3 mL of the solution to prepare W1. 2,400 mg of PLGA (LA:GA=50:50, Mw=20 kDa) and 15 mg of Span 85 are weighed and then dissolved in 10 mL of dichloromethane to prepare O. 500 mL of solution containing Tween 20 at 1.5 g/ 100 mL and sodium chloride at 0.2 g/ 100 mL is prepared to serve as an outer aqueous phase, and a pH value of the solution is adjusted to 7.8 to obtain W2. W1 is added to O and then dispersed by means of high-pressure homogenization at a pressure of 1000 bar for 2 min to form a primary emulsion. The primary emulsion is added to W2 under stirring to form a pre-double emulsion. The pre-double emulsion is poured into a rapid membrane emulsifier for emulsification and dispersion to obtain a double emulsion, where a used membrane tube has a pore size of 40 μm and a transmembrane pressure of 50 kPa. The double emulsion is solidified with a thin-film evaporator at 35 °C for 10 min, and the process is repeated for three times. After the solidification is finished, washing and lyophilizing are carried out to prepare a semaglutide-containing sustained-release microsphere.

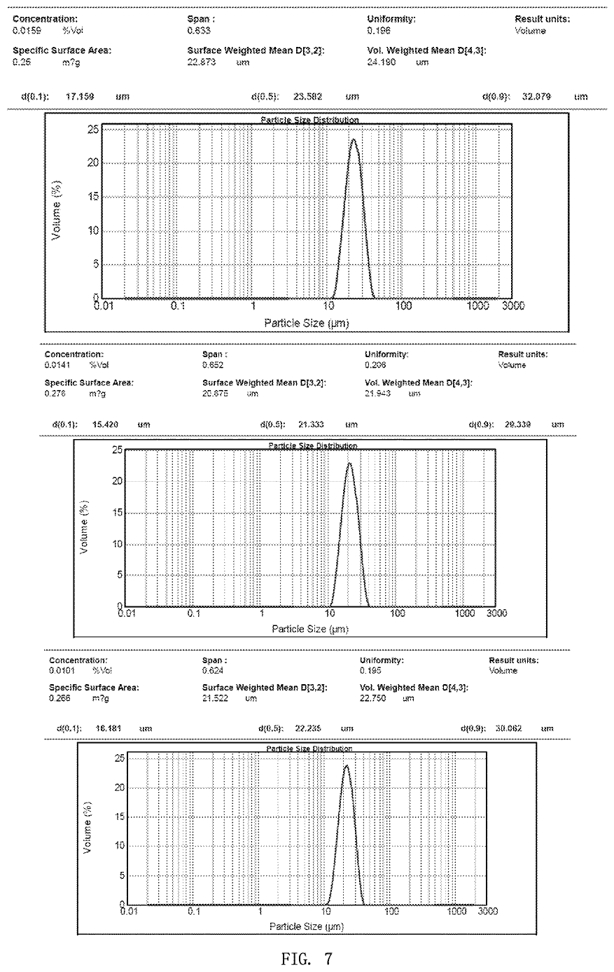

An appropriate amount of microspheres is weighed, and a small amount of deionized water is added thereto for dispersion to obtain a mixture, and the mixture is transferred to a laser particle size analyzer to determine a particle size of the microsphere, where an obtained particle size distribution diagram is shown in FIG. 7.

### Embodiment 2

30 mg of SNAC and 20 mg of poloxamer 188 are weighed and then dissolved in 10 mL of ultrapure water to obtain a solution, a pH value of the solution is adjusted to 7.0, and 400 mg of semaglutide is weighed and then dissolved in 3 mL of the solution to prepare W1. 2,000 mg of PLGA (LA GA=75:25, Mw=40 kDa) and 65 mg of soybean phospholipid are weighed and then dissolved in 10 mL of dichloromethane to prepare O. 200 mL of solution containing PVA at 1.5 g/ 100 mL and sodium chloride at 2.0 g/ 100 mL is prepared to serve as an outer aqueous phase, and a pH value of the solution is adjusted to 7.0 to obtain W2. W1 is added to O and homogenized for 5 min at an average rotational speed of 10,000 rpm to form a primary emulsion. The primary emulsion is added to W2 under stirring to form a pre-double emulsion. The pre-double emulsion is poured into a rapid membrane emulsifier for emulsification and dispersion to obtain a double emulsion, where a used membrane tube has a pore size of 20 μm and a transmembrane pressure of 78 kPa. The double emulsion is solidified by means of volatilization for 6 hours. After the solidification is finished, washing and lyophilizing are carried out to prepare a semaglutide-containing sustained-release microsphere.

### Embodiment 3

30 mg of phenol and 20 mg of poloxamer 188 are weighed and then dissolved in 10 mL of ultrapure water to obtain a solution, a pH value of the solution is adjusted to 7.0, and 400 mg of semaglutide is weighed and then dissolved in 3 mL of the solution to prepare W1. 2,000 mg of PLGA (LA:GA=75:25, Mw=40 kDa) and 65 mg of soybean phospholipid are weighed and then dissolved in 10 mL of dichloromethane to prepare O. 200 mL of solution containing PVA at 1.5 g/ 100 mL and sodium chloride at 2.0 g/ 100 mL is prepared to serve as an outer aqueous phase, and a pH value of the solution is adjusted to 7.0 to obtain W2. W1 is added to O and homogenized for 5 min at an average rotational speed of 10,000 rpm to form a primary emulsion. The primary emulsion is added to W2 under stirring to form a pre-double emulsion. The pre-double emulsion is poured into a rapid membrane emulsifier for emulsification and dispersion to obtain a double emulsion, where a used membrane tube has a pore size of 20 μm and a transmembrane pressure of 78 kPa. The double emulsion is solidified by means of volatilization for 6 hours. After the solidification is finished, washing and lyophilizing are carried out to prepare a semaglutide-containing sustained-release microsphere.

### Embodiment 4

50 mg of benzyl alcohol and 50 mg of Tween 20 are weighed and then dissolved in 10 mL of ultrapure water to obtain a solution, a pH value of the solution is adjusted to 7.4, and 150 mg of semaglutide is weighed and then dissolved in 3 mL of the solution to prepare W1. 2,000 mg of PLGA (LA:GA=50:50, Mw=5 kDa) and 300 mg of lecithin are weighed and then dissolved in 10 mL of dichloromethane to prepare O. 250 mL of solution containing PVA at 1.0 g/ 100 mL and sodium chloride at 2.0 g/ 100 mL is prepared to serve as an outer aqueous phase, and a pH value of the solution is adjusted to 7.0 to obtain W2. W1 is added to O and homogenized for 5 min at an average rotational speed of 12,000 rpm to form a primary emulsion. The primary emulsion is added to W2 under stirring to form a pre-double emulsion. The pre-double emulsion is poured into a rapid membrane emulsifier for emulsification and dispersion to obtain a double emulsion, where a used membrane tube has a pore size of 30 μm and a transmembrane pressure of 62 kPa. The double emulsion is solidified by means of volatilization for 6 hours. After the solidification is finished, washing and lyophilizing are carried out to prepare a semaglutide-containing sustained-release microsphere.

### Comparative Example 1

90 mg of sucrose is weighed and then dissolved in 10 mL of ultrapure water to obtain a solution, a pH value of the solution is adjusted to 7.6, and 70 mg semaglutide is weighed and then dissolved in 3 mL of solution to obtain W1. 2,400 mg PLGA (LA:GA=50:50, Mw=20 kDa) is weighed in 10 mL of dichloromethane to prepare O. 500 mL of solution containing Tween 20 at 1.5 g/ 100 mL is prepared to serve as an outer aqueous phase, and a pH value of the solution is adjusted to 7.8 to obtain W2. W1 is added to O and dispersed by high-pressure homogenization at a pressure of 1000 bar for 2 min to form a primary emulsion. The primary emulsion is added to W2 under stirring to form a pre-double emulsion. The pre-double emulsion is poured into a rapid membrane emulsifier for emulsification and dispersion to obtain a double emulsion, where a used membrane tube has a pore size of 40 μm and a transmembrane pressure of 50 kPa. The double emulsion is solidified with a thin-film evaporator at 35 °C for 10 min, and the process is repeated for three times. After the solidification is finished, washing and lyophilizing are carried out to prepare a semaglutide-containing sustained-release microsphere.

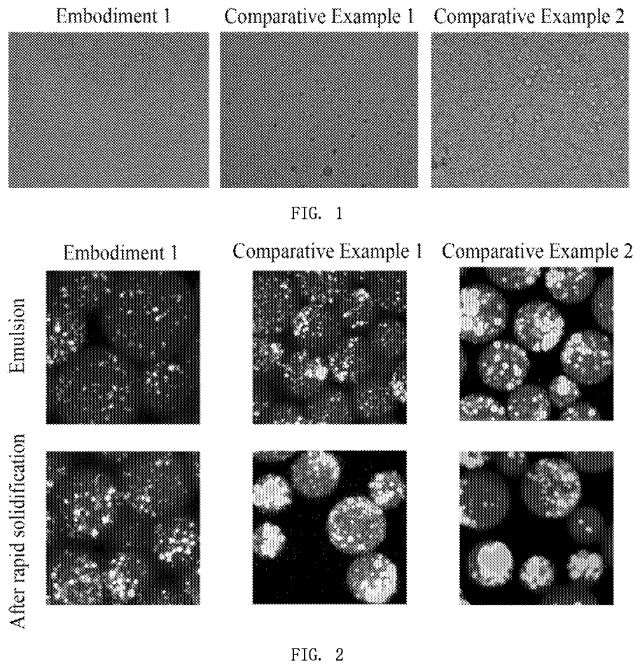

Two emulsion stabilizers are added in Embodiment 1, while no emulsion stabilizer is added in Comparative Example 1. It can be seen from FIG. 1 that the primary emulsion of the semaglutide-containing sustained-release microsphere in Embodiment 1 presents fine particle sizes, and can be densely distributed in the oil phase with a small particle size, while the primary emulsion of semaglutide-containing sustained-release microsphere in Comparative Example has large and nonuniform particle sizes, indicating that the addition of the emulsion stabilizer in the preparation process of the present disclosure is essential for forming the uniformly dispersed primary emulsion.

### Comparative Example 2

90 mg of sucrose is weighed and then dissolved in 10 mL of ultrapure water to obtain a solution, and 70 mg semaglutide is weighed and then dissolved in 3 mL of solution to obtain W1. 2,400 mg PLGA (LA:GA=50:50, Mw=20 kDa) and 60 mg of Span 85 are weighed and then dissolved in 10 mL of dichloromethane to prepare O. 500 mL of solution containing Tween 20 at 1.5 g/ 100 mL is prepared to serve as an outer aqueous phase, and a pH value of the solution is adjusted to 7.8 to obtain W2. W1 is added to O and dispersed by high-pressure homogenization at a pressure of 1000 bar for 2 min to form a primary emulsion. The primary emulsion is added to W2 under stirring to form a pre-double emulsion. The pre-double emulsion is poured into a rapid membrane emulsifier for emulsification and dispersion to obtain a double emulsion, where a used membrane tube has a pore size of 40 μm and a transmembrane pressure of 50 kPa. The double emulsion is solidified with a thin-film evaporator at 35 °C for 10 min, and the process is repeated for three times. After the solidification is finished, washing and lyophilizing are carried out to prepare a semaglutide-containing sustained-release microsphere.

Two emulsion stabilizers are added in Embodiment 1, while merely one emulsion stabilizer is added in Comparative Example 2. It can be seen from FIG. 1 that compared with the fine and uniform primary emulsion of the primary emulsion of the semaglutide-containing sustained-release microsphere in Embodiment 1, the primary emulsion of Comparative Example 2 presents nonuniform distribution. Droplets coalesce rapidly after emulsion preparation, leading to formation of a large number of large chambers inside the emulsion system, indicating that that the addition of a single emulsion stabilizer fails to achieve uniform emulsion distribution.

### Comparative Example 3

70 mg of semaglutide is weighed and then dissolved in 3 mL ultra-pure water to obtain W1, and a pH value of the solution is adjusted to 7.6. 2,400 mg PLGA (LA:GA=50:50, Mw=20 kDa) and 15 mg of Span 85 are weighed and then dissolved in 10 mL of dichloromethane to prepare O. 500 mL of solution containing Tween 20 at 1.5 g/ 100 mL is prepared to serve as an outer aqueous phase, and a pH value of the solution is adjusted to 7.8 to obtain W2. W1 is added to O and dispersed by means of high-pressure homogenization at a pressure of 1000 bar for 2 min to form a primary emulsion. The primary emulsion is added to W2 under stirring to form a pre-double emulsion. The pre-double emulsion is poured into a rapid membrane emulsifier for emulsification and dispersion to obtain a double emulsion, where a used membrane tube has a pore size of 40 μm and a transmembrane pressure of 50 kPa. The double emulsion is solidified with a thin-film evaporator at 35 °C for 10 min, and the process is repeated for three times. After the solidification is finished, washing and lyophilizing are carried out to prepare a semaglutide-containing sustained-release microsphere.

### Embodiment 5: Characterization of Emulsion Solidification and Formation

In this embodiment, the semaglutide-containing sustained-release microspheres prepared in the foregoing Embodiment 1, Comparative Example 1 and Comparative Example 2 are respectively taken as research objects for a characterization test. The specific methods are as follows:

FITC (Fluorescein Isothiocyanate) is mixed with a drug substance and incubated overnight for labeling, and unlabeled FITC is then removed by means of ultrafiltration washing to obtain a mixture. The mixture is lyophilized in the dark to obtain fluorescently labeled drug substance for subsequent microsphere preparation. Nile red is mixed with the oil phase solution to label the carrier material, thereby characterizing drug distribution during emulsion formation and microsphere solidification. The experimental results are shown in FIG. 2.

It can be seen from FIG. 2 that in the semaglutide-containing sustained-release microspheres of Embodiment 1, the semaglutide is densely distributed in the emulsion in the form of fine droplets. The emulsion stability can be maintained during rapid solidification, and there is no drug enrichment on an outer shell of the microsphere. In Comparative Example 1 and Comparative Example 2, the primary emulsion exhibits non-uniform particle size and irregular drug distribution. During solidification, the dispersed primary emulsion droplets coalesce in large quantities and migrate to the outer surface of microsphere, indicating that the emulsion stabilizer added during the preparation can avoid burst release and has long-term sustained-release performance.

Embodiment 6: In Vitro Release Test of Microspheres

In this embodiment, the semaglutide-containing sustained-release microspheres prepared in the foregoing Embodiment 1, Comparative Example 1, 1 and Comparative Example 3 are respectively taken as examples for in vitro release test. The specific methods are as follows.

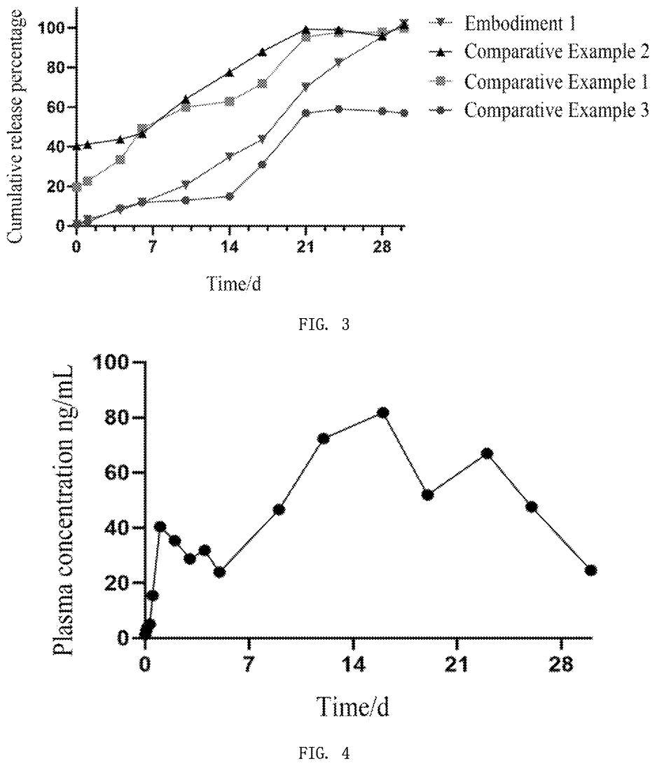

An appropriate amount of the sample (equivalent to approximately 2 mg of semaglutide) is placed into a 50 mL centrifuge tube, mixed thoroughly with 10 mL of commercially available 0.01 M PBS (Phosphate-buffered Saline) serving as a release medium to form a mixture, and the centrifuge tube is then incubated in a water bath at 37.5 °C. At predetermined time points of 0.5 h, 24 h, 3 d, 6 d, 10 d, 14 d, 17 d, 21 d, 24 d, 28 d and 30 d, the mixture is centrifuged at 8,000 rpm for 5 min; 1 mL of supernatant is precisely pipetted, and 1 mL of release medium is precisely supplemented subsequently. The mixture is dispersed evenly and continuously shaken. A sample solution at each time point is taken and then filtered through a 0.45 μm polyvinylidene fluoride (PVDF) membrane for collecting a subsequent filtrate, thereby obtaining a test solution for drug content determination. The experimental results are shown in FIG. 3.

FIG. 3 shows that the semaglutide-containing sustained-release microspheres of Comparative Examples 1 and 2 have higher burst release, and the semaglutide-containing sustained-release microsphere in Comparative Example 2 exhibits an initial release lag, and has subsequent sustained release as excipients degrade, with an early attainment of the release endpoint. In contrast, the semaglutide-containing sustained-release microsphere prepared in Comparative Example 3 exhibits a lower burst release, but a relatively faster release rate in the mid phase and a lower release at the endpoint, with a release rate far below that of Embodiment 1 and Comparative Examples 1 and 2. The semaglutide-containing sustained-release microsphere in Embodiment 1 shows no apparent burst release, and stable release behavior in the mid-to late-stage, indicating that the semaglutide-containing sustained-release microsphere prepared in the present disclosure has long-term sustained release.

### Embodiment 7: In Vivo PK Testing in Animals

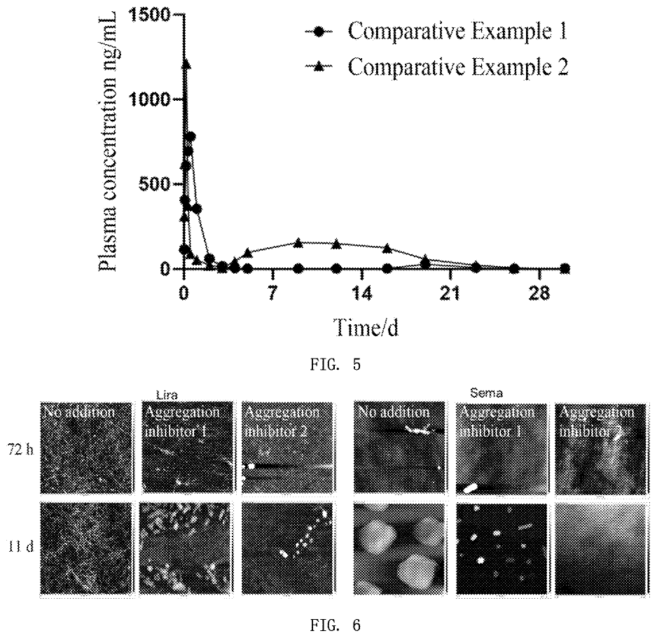

In this embodiment, the semaglutide-containing sustained-release microspheres prepared in the foregoing Embodiment 1, Comparative Example 1, and Comparative Example 2 are respectively taken as examples for animal PK testing, with specific methods as follows:
(1) Animal screening and grouping: twelve male SD rats are screened and divided equally into three groups by body weight, with four rats in each group. Group 1: Embodiment 1, Group 2: Comparative Example 1, Group 3: Comparative Example 2.
(2) Administration frequency and route: All rats in each group are administrated a single subcutaneous dose of a test article solution at 1 mg/kg, sequentially by group.
(3) Blood sampling time points: Blood samples are collected once before administration, and at 0.5 h, 2 h, 4 h, 8 h, 12 h, 24 h, 2 d, 3 d, 4 d, 5 d, 9 d, 12 d, 16 d, 19 d, 23 d, 26 d, and 30 d after administration after administration.
(4) Blood sampling method: Blood samples from all groups are collected via jugular vein puncture.
(5) Detection Method: a method for detecting a plasma concentration of semaglutide in the rat includes carrying out protein precipitation extraction after adding an isotopically labeled internal standard to a calibration curve samples, a quality control sample, and an unknown (test) sample, and then carrying out LC-MS/MS detection, where PK curves of the rats are shown in FIG. 4 (Embodiment 1) and FIG. 5 (Comparative Examples 1 and 2).

It can be seen from FIG. 4 and FIG. 5 that the plasma concentration of the semaglutide-containing sustained-release microspheres prepared in Comparative Example 1 and Comparative Example 2 of the present disclosure decrease rapidly after dosing. Although Comparative Example 2 shows a slight increase in plasma concentration in the second week after dosing, the overall concentration remains at a low level. In contrast, the semaglutide-containing sustained-release microsphere of Embodiment 1 exhibits no apparent burst release. The plasma concentration reaches its peak on the fifteenth day and remains within a relatively high concentration range for 30 days, indicating that the microsphere prepared by the present disclosure has low burst release and excellent long-term sustained-release performance.

### Embodiment 8: Effect Simulation of Aggregation Inhibitor

In this embodiment, simulation experiments of anti-aggregation effects are carried out using liraglutide (Lira) and semaglutide (Sema) as examples, respectively, with specific methods as follows.

A mixture of aggregation inhibitors (aggregation inhibitor 1 is sucrose, and aggregation inhibitor 2 is phenol), a drug substance, and deionized water are mixed at a weight ratio of 1:3:100 and subsequently lyophilized. PBS (pH 7.4) is added dropwise to the lyophilized drug powder to simulate gradual infiltration of the drug inside THE microsphere. Afterwards, a portion of the drug is dissolved in the PBS, and an atomic force microscope (AFM) is used to observe whether fibrils are formed by the drug and to simulate structural changes of the drug during the slow permeation process inside the microsphere. The aggregation results are shown in FIG. 6.

It can be seen from FIG. 6 that the aggregation inhibitor can significantly inhibit fibrillation of liraglutide and semaglutide, indicating that the preparation method provided by the present disclosure can effectively inhibit drug aggregation into fibrils. The semaglutide-containing sustained-release microsphere prepared by the present disclosure can effectively avoid the loss of drug activity.

It should be noted that although the technical solution of the present disclosure is introduced with specific instances, those skilled in the art may understand that the present disclosure should not be limited thereto.

Various embodiments of the present disclosure have been described above. The foregoing description is illustrative, non-exhaustive, and not limited to the disclosed embodiments. Many modifications and variations are apparent to those of ordinary skill in the art without departing from the scope and spirit of the described embodiments. The selection of these terms used herein is intended to best illustrate the principles of the embodiments, practical applications, or technical improvements in the industry, and to enable other persons skilled in the art to readily understand the embodiments disclosed herein.

1. A preparation method for a polypeptide drug-containing sustained-release microsphere, comprising the following steps:
mixing an aggregation inhibitor, a first emulsion stabilizer, and water to obtain a mixture, regulating a pH value of the mixture with a first pH regulator, and then adding the polypeptide drug into the mixture to form an inner aqueous phase;
mixing a carrier material, a second emulsion stabilizer, and an organic solvent to form an oil phase;
mixing a surfactant, an osmotic pressure regulator, and water to obtain a mixture, regulating a pH value of the mixture with a second pH regulator to form an outer aqueous phase; and
dispersing the inner aqueous phase into the oil phase to form a primary emulsion, dispersing the primary emulsion into the outer aqueous phase to form a pre-double emulsion, emulsifying the pre-double emulsion to form a double emulsion, and solidifying the double emulsion to obtain the sustained-release microsphere.

2. The preparation method according to claim 1, wherein the preparation method uses one or more substances selected from the group consisting of:
the polypeptide drug is a peptide GLP-1R agonist, comprising at least one of liraglutide and semaglutide;
the aggregation inhibitor is at least one of sodium caprylate, sucrose, mannitol, sodium 8-(2-hydroxybenzamido)octanoate, phenol, benzyl alcohol, ethanol, and trifluoroethanol, a weight ratio of the aggregation inhibitor to the polypeptide drug is 1:70 to 50:70;
the first emulsion stabilizer is at least one of Tween, poloxamer, sodium oleate, and dextran;
a weight ratio of the emulsion stabilizer A to the polypeptide drug is 1:200 to 3:2;
the water is at least one of ultra-pure water, double-distilled water, and water for injection; a dosage ratio of the water to the polypeptide drug is 3 mL:10 to 650 mg; and
the pH regulator A is at least one of hydrogen chloride, sodium hydroxide, and phosphate; a pH value of the mixture is adjusted to 6.0 to 9.5 by the first pH regulator.

3. The preparation method according to claim 1, wherein the preparation method uses one or more substances selected from the group consisting of:
the organic solvent is at least one of trichloromethane, dichloromethane, ethyl acetate, acetone, and acetonitrile; a dosage ratio of the organic solvent to the polypeptide drug is 1 to 10 mL:50 mg;
the carrier material is a biodegradable carrier material, the carrier material is at least one of poly(lactic-co-glycolic acid), poly(lactic acid)-poly(ethylene glycol) copolymer, polylactic acid, polyhydroxyalkanoate, and polycaprolactone, a ratio of lactic acid to glycolic acid in every 100 parts of monomers is 100 to 50:0 to 50, poly(lactic-co-glycolic acid) in 50:50; a molecular weight of the carrier material is 1000 to 150,000 Da; a weight ratio of the carrier material to the polypeptide drug is 3:1 to 100:1; and
the second emulsion stabilizer is at least one of lecithin, soybean phospholipid, oleic acid, and Span, a weight ratio of the emulsion stabilizer B to the polypeptide drug is 1:20 to 40:20.

4. The preparation method according to claims 1, wherein the preparation method uses one or more substances selected from the group consisting of:
the surfactant is at least one of polyvinyl alcohol, Tween, sodium dodecyl sulfate, sodium dodecyl benzene sulfonate, and polyoxyethylene octylphenol ether, a weight ratio of the surfactant to the polypeptide drug is 1:20 to 40:20;
the osmotic pressure regulator is at least one of sodium chloride and potassium chloride; a weight ratio of the osmotic pressure regulator to the polypeptide drug is 1:1 to 100:1;
the water is at least one of ultra-pure water, double-distilled water, and water for injection; a dosage ratio of the water to the polypeptide drug is 0.001 to 10 mL:1 mg; and
the second pH regulator is at least one of hydrogen chloride, sodium hydroxide, and phosphate; and/or a pH value of the mixture is adjusted to 6.0 to 9.5 by the second pH regulator.

5. The preparation method according to any one of claims 1, wherein the preparation method uses one or more substances selected from the group consisting of:
the dispersion is accomplished by means of at least one of stirring, homogenization, and ultrasonication; the high-pressure homogenization lasts for 0.5 to 20 min; the high-pressure homogenization is carried out at a pressure of 0.2 to 1200 bar;
the redispersion is accomplished by means of at least one of stirring and homogenization;
the emulsification is accomplished by means of membrane emulsification, a membrane pore size for the membrane emulsification is 1.0 to 60 μm; the membrane emulsification is carried out at a transmembrane pressure of 1 to 200 kPa; and
the solidification is accomplished by means of volatilization or evaporation, reduced-pressure volatilization or thin-film evaporation, the thin-film evaporation lasts for 1 to 50 min; the thin-film evaporation is carried out at a temperature of 25 to 50 °C; a number of cycles for the thin-film evaporation is 1 to 6.

6. The preparation method according to any one of claims 1, wherein the preparation method further comprises the following steps:
solidifying the double emulsion, followed by washing and lyophilizing.

7. The preparation method according to any one of claims 1, wherein the preparation method uses one or more substances selected from the group consisting of:
the sustained-release microsphere has a particle size of 1.0 to 100 μm; and a particle size distribution span (Span) is 0.2 to 2.0;
encapsulation efficiency of the sustained-release microsphere is 80% to 105%; and
a drug loading capacity of the sustained-release microsphere is 0.5% to 25%.

8. A polypeptide drug-containing sustained-release microsphere, wherein the sustained-release microsphere is prepared by a preparation method comprising:
mixing an aggregation inhibitor, a first emulsion stabilizer, and water to obtain a mixture, regulating a pH value of the mixture with a first pH regulator, and then adding the polypeptide drug into the mixture to form an inner aqueous phase;
mixing a carrier material, a second emulsion stabilizer, and an organic solvent to form an oil phase;
mixing a surfactant, an osmotic pressure regulator, and water to obtain a mixture, regulating a pH value of the mixture with a second pH regulator to form an outer aqueous phase; and
dispersing the inner aqueous phase into the oil phase to form a primary emulsion, dispersing the primary emulsion into the outer aqueous phase to form a pre-double emulsion, emulsifying the pre-double emulsion to form a double emulsion, and solidifying the double emulsion to obtain the sustained-release microsphere.

9. The polypeptide drug-containing sustained-release microsphere according to claim 8,
wherein the preparation method uses one or more substances selected from the group consisting of:
the polypeptide drug is a peptide GLP-1R agonist, comprising at least one of liraglutide and semaglutide;
the aggregation inhibitor is at least one of sodium caprylate, sucrose, mannitol, sodium 8-(2-hydroxybenzamido)octanoate, phenol, benzyl alcohol, ethanol, and trifluoroethanol, a weight ratio of the aggregation inhibitor to the polypeptide drug is 1:70 to 50:70;
the first emulsion stabilizer is at least one of Tween, poloxamer, sodium oleate, and dextran;
a weight ratio of the emulsion stabilizer A to the polypeptide drug is 1:200 to 3:2;
the water is at least one of ultra-pure water, double-distilled water, and water for injection; a dosage ratio of the water to the polypeptide drug is 3 mL:10 to 650 mg; and
the pH regulator A is at least one of hydrogen chloride, sodium hydroxide, and phosphate; a pH value of the mixture is adjusted to 6.0 to 9.5 by the first pH regulator.

10. The polypeptide drug-containing sustained-release microsphere according to claim 8,
wherein the preparation method uses one or more substances selected from the group consisting of:
the organic solvent is at least one of trichloromethane, dichloromethane, ethyl acetate, acetone, and acetonitrile; a dosage ratio of the organic solvent to the polypeptide drug is 1 to 10 mL:50 mg;
the carrier material is a biodegradable carrier material, the carrier material is at least one of poly(lactic-co-glycolic acid), poly(lactic acid)-poly(ethylene glycol) copolymer, polylactic acid, polyhydroxyalkanoate, and polycaprolactone, a ratio of lactic acid to glycolic acid in every 100 parts of monomers is 100 to 50:0 to 50, poly(lactic-co-glycolic acid) in 50:50; a molecular weight of the carrier material is 1000 to 150,000 Da; a weight ratio of the carrier material to the polypeptide drug is 3:1 to 100:1; and
the second emulsion stabilizer is at least one of lecithin, soybean phospholipid, oleic acid, and Span, a weight ratio of the emulsion stabilizer B to the polypeptide drug is 1:20 to 40:20.

11. The polypeptide drug-containing sustained-release microsphere according to claim 8,
wherein the preparation method uses one or more substances selected from the group consisting of:
the surfactant is at least one of polyvinyl alcohol, Tween, sodium dodecyl sulfate, sodium dodecyl benzene sulfonate, and polyoxyethylene octylphenol ether, a weight ratio of the surfactant to the polypeptide drug is 1:20 to 40:20;
the osmotic pressure regulator is at least one of sodium chloride and potassium chloride; a weight ratio of the osmotic pressure regulator to the polypeptide drug is 1:1 to 100:1;
the water is at least one of ultra-pure water, double-distilled water, and water for injection; a dosage ratio of the water to the polypeptide drug is 0.001 to 10 mL:1 mg; and
the second pH regulator is at least one of hydrogen chloride, sodium hydroxide, and phosphate; and/or a pH value of the mixture is adjusted to 6.0 to 9.5 by the second pH regulator.

12. The polypeptide drug-containing sustained-release microsphere according to claim 8,
wherein the preparation method uses one or more substances selected from the group consisting of:
the dispersion is accomplished by means of at least one of stirring, homogenization, and ultrasonication; the high-pressure homogenization lasts for 0.5 to 20 min; the high-pressure homogenization is carried out at a pressure of 0.2 to 1200 bar;
the redispersion is accomplished by means of at least one of stirring and homogenization;
the emulsification is accomplished by means of membrane emulsification, a membrane pore size for the membrane emulsification is 1.0 to 60 μm; the membrane emulsification is carried out at a transmembrane pressure of 1 to 200 kPa;
the solidification is accomplished by means of volatilization or evaporation, reduced-pressure volatilization or thin-film evaporation, the thin-film evaporation lasts for 1 to 50 min; the thin-film evaporation is carried out at a temperature of 25 to 50 °C; a number of cycles for the thin-film evaporation is 1 to 6.

13. The polypeptide drug-containing sustained-release microsphere according to claim 8, the preparation method further comprises the following steps:
solidifying the double emulsion, followed by washing and lyophilizing.

14. The polypeptide drug-containing sustained-release microsphere according to claim 8, wherein the preparation method uses one or more substances selected from the group consisting of:
the sustained-release microsphere has a particle size of 1.0 to 100 μm; and a particle size distribution span (Span) is 0.2 to 2.0;
encapsulation efficiency of the sustained-release microsphere is 80% to 105%; and
a drug loading capacity of the sustained-release microsphere is 0.5% to 25%.

15. A pharmaceutical formulation of a polypeptide drug, wherein the pharmaceutical formulation comprises the sustained-release microsphere according to claim 8.

16. The pharmaceutical formulation of a polypeptide drug according to claim 15, wherein
the pharmaceutical formulation further comprises at least one pharmaceutically acceptable excipient.

17. The pharmaceutical formulation of a polypeptide drug according to claim 15, wherein the pharmaceutical formulation is a sustained-release formulation.

18. The pharmaceutical formulation of a polypeptide drug according to claim 15, wherein the pharmaceutical formulation is an injection.
